(19) United States
(12) Patent Application Publication
(10) Pub. No.: US 2022/0073867 A1
(43) Pub. Date: Mar. 10, 2022

(54) CELL CULTURE AUXILIARY AGENT AND CELL CULTURE MEDIUM USING THE SAME

(71) Applicant: 3D GLOBAL BIOTECH INC., NEW TAIPEI CITY (TW)

(72) Inventor: Keng-Liang Ou, New Taipei City (TW)

(21) Appl. No.: 17/458,558

(22) Filed: Aug. 27, 2021

(30) Foreign Application Priority Data
Sep. 8, 2020 (TW) 109130694

(51) Int. Cl. C12N 5/00

(52) U.S. Cl. CPC C12N 5/0062; C12N 5/0075; C12N 2531/00; C12N 2539/10; C12N 2513/00; C12N 2533/30

(57) ABSTRACT

A cell culture auxiliary agent and a cell culture medium using the same are provided. The cell culture auxiliary agent is formed by attaching each coordination peptide having cell affinity to two side ends of a polyoxyethylene polyoxypropylene ether block copolymer through anhydride monomers.

### CROSS-REFERENCE TO RELATED PATENT APPLICATION

This application claims the benefit of priority to Taiwan Patent Application No. 109130694, filed on Sep. 8, 2020. The entire content of the above identified application is incorporated herein by reference.

Some references, which may include patents, patent applications and various publications, may be cited and discussed in the description of this disclosure. The citation and/or discussion of such references is provided merely to clarify the description of the present disclosure and is not an admission that any such reference is “prior art” to the disclosure described herein. All references cited and discussed in this specification are incorporated herein by reference in their entireties and to the same extent as if each reference was individually incorporated by reference.

### FIELD OF THE DISCLOSURE

The present disclosure relates to a cell culture auxiliary agent, and more particularly to a cell culture auxiliary agent capable of providing three-dimensional (3D) culture conditions, and a cell culture medium using the same.

### BACKGROUND OF THE DISCLOSURE

Generally, using a cell culture medium for cell culture, the cells will settle on a certain area of the cell culture device due to gravity, thus being in a 2D cell culture condition.

With the development of technology, scientists began to use 3D cell culture technology to replace 2D cell culture technology to reflect the growth condition of cells in vivo. Conventional 3D cell culture techniques include hanging-drop culture, 3D cell culture scaffolds (prefabricated scaffolds), and gel-embedded culture, among which gel-embedded culture is the most common.

However, compared with general cell culture medium culture, the use of colloids for 3D culture requires additional preparation of colloids, which leads to prolonged experiment time and increased experiment costs. In addition, in the 3D culture environment formed by solid colloids, as the cells gradually gather together, nutrients and gases will be difficult to transmit to the center of the tissue, and it is easy to cause cell necrosis, in this state, long-term cell culture cannot be implemented. Further, when the cell culture is completed, the cells embedded in the colloid are not easy to take out, which causes burdens on the operation of the experimenter and may damage the integrity of the cells.

Therefore, how to improve the cell culture medium so that the environment of in vitro cell culture can be close to the growth environment of cells in vivo and facilitate the operation of experimenters is an important urgent issue yet to be solved in this field.

### SUMMARY OF THE DISCLOSURE

In response to the above-referenced technical inadequacies, the present disclosure provides a cell culture auxiliary agent to create a three-dimensional culture environment. In addition, the present disclosure also provides a cell culture medium using the cell culture auxiliary agent.

In one aspect, the present disclosure provides a cell culture auxiliary agent, which includes a structural formula represented as follow: P-L-S-L-P. In the structural formula, S is a substrate, each L is a linker, and each P is a peptide. In addition, the substrate is a polyoxyethylene polyoxypropylene ether block copolymer, the linkers are each independently an anhydride monomer, and an amino acid sequence of the peptides are each independently selected from a group consisting of glycine-arginine-glycine-aspartate (Gly-Arg-Gly-Asp, GRGD), arginine-glycine-aspartate (Arg-Gly-Asp, RGD), arginine-glutamate-aspartate-valine (Arg-Glu-Asp-Val, REDV), leucine-aspartate-valine (Leu-Asp-Val, LDV), tyrosine-isoleucine-glycine-serine-arginine (Tyr-Ile-Gly-Ser-Arg, YIGSR), proline-aspartate-serine-glycine-arginine (Pro-Asp-Ser-Gly-Arg, PDSGR), isoleucine-lysine-valine-alanine-valine (Ile-Lys-Val-Ala-Val, IKVAV), and arginine-asparagine-isoleucine-alanine-glutamate-isoleucine-isoleucine-lysine-aspartate-alanine (Arg-Asn-Ile-Ala-Glu-Ile-Ile-Lys-Asp-Ala, RNIAEIIKDA).

In another aspect, the present disclosure provides cell culture medium, which includes 0.5-5 wt % of a cell culture auxiliary agent including a structural formula represented as follow: P-L-S-L-P. In the structural formula, S is a substrate, each L is a linker, and each P is a peptide. In addition, the substrate is a polyoxyethylene polyoxypropylene ether block copolymer, the linkers are each independently an anhydride monomer, and an amino acid sequence of the peptides are each independently selected from a group consisting of glycine-arginine-glycine-aspartate (Gly-Arg-Gly-Asp, GRGD), arginine-glycine-aspartate (Arg-Gly-Asp, RGD), arginine-glutamate-aspartate-valine (Arg-Glu-Asp-Val, REDV), leucine-aspartate-valine (Leu-Asp-Val, LDV), tyrosine-isoleucine-glycine-serine-arginine (Tyr-Ile-Gly-Ser-Arg, YIGSR), proline-aspartate-serine-glycine-arginine (Pro-Asp-Ser-Gly-Arg, PDSGR), isoleucine-lysine-valine-alanine-valine (Ile-Lys-Val-Ala-Val, IKVAV), and arginine-asparagine-isoleucine-alanine-glutamate-isoleucine-isoleucine-lysine-aspartate-alanine (Arg-Asn-Ile-Ala-Glu-Ile-Ile-Lys-Asp-Ala, RNIAEIIKDA).

In certain embodiments, the cell culture medium further includes 95-99.5 wt % of a cell culture solution.

In certain embodiments, the cell culture auxiliary agent is applied to make cells be suspended in the cell culture medium.

In certain embodiments, the substrate is Pluronic® F-127 (F127), and the anhydride monomer is maleic anhydride (MA), succinic anhydride, or 4-methacryloxyethyl trimellitic anhydride (4META).

In certain embodiments, the anhydride monomer is maleic anhydride, and the amino acid sequence of the peptide is glycine-arginine-glycine-aspartate (Gly-Arg-Gly-Asp, GRGD).

In certain embodiments, the anhydride monomer is 4-methacryloxyethyl trimellitic anhydride (4META), and the amino acid sequence of the peptide is glycine-arginine-glycine-aspartate (Gly-Arg-Gly-Asp, GRGD).

Therefore, by virtue of “the cell culture auxiliary agent is formed by attaching each coordination peptide having cell affinity to two side ends of a polyoxyethylene polyoxypropylene ether block copolymer through anhydride monomers”, the cell culture auxiliary agent of the present disclosure can form a microstructure carrier that allows cells to attach in the cell culture medium, and allows the cells to grow in a suspended state in the cell culture medium, thereby improving cell proliferation and survival rate.

Furthermore, because the polyoxyethylene polyoxypropylene ether block copolymer is temperature-sensitive, the cell culture substrate of the present disclosure can form a hydrogel-like microstructure carrier at room temperature (about 25° C.) or culture temperature (about 37° C.), which can carry cells and keep the cells in a dispersed and suspended state, and then aggregate in a spherical shape after division. It is worth noting that the microstructure carriers can be turned back into liquid at low temperature (below 10° C.), which is convenient for cell collection or replacement of cell culture medium. Therefore, the cell culture medium of the present disclosure can provide cells cultured in vitro having basically the same growth conditions as cells cultured in vivo, and make the operation more convenient and flexible for experimenters; and can reduce the experiment cost because no additional 3D colloid is required.

Moreover, the cell culture auxiliary agent of the present disclosure use anhydride monomers to link polyoxyethylene polyoxypropylene ether block copolymers with cell-affinity peptides, which can improve structural stability, and the peptides can more easily help cells aggregate and suspend.

These and other aspects of the present disclosure will become apparent from the following description of the embodiment taken in conjunction with the following drawings and their captions, although variations and modifications therein may be affected without departing from the spirit and scope of the novel concepts of the disclosure.

### BRIEF DESCRIPTION OF THE DRAWINGS

The described embodiments may be better understood by reference to the following description and the accompanying drawings, in which:

FIG. 1 is a schematic view of a cell culture medium according to a second embodiment of the present disclosure;

FIG. 2 is a microscopic image of a cell culture result according to a third embodiment of the present disclosure;

FIG. 3 is a bar graph of cell viability according to the third embodiment of the present disclosure;

FIG. 4 is a line graph of cell viability (absorbance value) according to the third embodiment of the present disclosure;

FIG. 5 is a microscopic image of a cell culture result according to a fourth embodiment of the present disclosure; and

FIG. 6 is a bar graph of cell viability according to the fourth embodiment of the present disclosure.

### DETAILED DESCRIPTION OF THE EXEMPLARY EMBODIMENTS

The present disclosure is more particularly described in the following examples that are intended as illustrative only since numerous modifications and variations therein will be apparent to those skilled in the art. Like numbers in the drawings indicate like components throughout the views. As used in the description herein and throughout the claims that follow, unless the context clearly dictates otherwise, the meaning of “a”, “an”, and “the” includes plural reference, and the meaning of “in” includes “in” and “on”. Titles or subtitles can be used herein for the convenience of a reader, which shall have no influence on the scope of the present disclosure.

The terms used herein generally have their ordinary meanings in the art. In the case of conflict, the present document, including any definitions given herein, will prevail. The same thing can be expressed in more than one way. Alternative language and synonyms can be used for any term(s) discussed herein, and no special significance is to be placed upon whether a term is elaborated or discussed herein. A recital of one or more synonyms does not exclude the use of other synonyms. The use of examples anywhere in this specification including examples of any terms is illustrative only, and in no way limits the scope and meaning of the present disclosure or of any exemplified term. Likewise, the present disclosure is not limited to various embodiments given herein. Numbering terms such as “first”, “second” or “third” can be used to describe various components, signals or the like, which are for distinguishing one component/signal from another one only, and are not intended to, nor should be construed to impose any substantive limitations on the components, signals or the like.

### First Embodiment

A first embodiment of the present disclosure provides a cell culture auxiliary agent, which includes a structural formula represented as follows: P-L-S-L-P. In the structural formula, S is a substrate, each L is a linker, and each P is a peptide. In operation, the dosage form of the cell culture auxiliary agent of the present disclosure can be powder or concentrated solution, but the present disclosure is not limited thereto.

Further, the substrate in the cell culture auxiliary agent of the present disclosure is a polyoxyethylene polyoxypropylene ether block copolymer, and more specifically is a non-ionic triblock copolymer formed by binding a hydrophilic polyoxyethylene on two sides of the hydrophobic polyoxypropylene. The length of the polyoxyethylene ether block copolymer can be adjusted according to particular implementations, so there are slightly different types of properties. In this embodiment, the substrate is Pluronic® F-127 (F127), which is a temperature-sensitive material, it is liquid when it is in a low temperature environment (about 4-10° C.), it is hydrogel-like when it is close to room temperature (about 25° C.) or body temperature (about 37° C.), and can be transformed back to liquid at low temperature.

The linkers in the cell culture auxiliary agent of the present disclosure are each independently an acid anhydride monomer, and are respectively connected to the two hydrophilic ends of the polyoxyethylene polyoxypropylene ether block copolymer. In this embodiment, each of the linkers can be maleic anhydride (MA), succinic anhydride, or 4-methacryloxyethyl trimellitic anhydride (4META), but the present disclosure is not limited thereto. Each of the linkers is used to stably bind the substrate and the peptide together. In view of structural stability and reliability, each of the linkers is 4-methacryloxyethyl trimellitic anhydride (4META) or maleic anhydride, and more preferably maleic anhydride.

It should be noted that because 4-methacryloyloxyethyl trimellitic anhydride has a larger molecular structure (molecular weight of 304.25), the distance between the substrate and the peptide increases, resulting in fewer peptides in the same unit volume. In contrast, maleic anhydride has a smaller molecular structure (molecular weight of 98.06), which can shorten the distance between the substrate and the peptide, thereby increasing the number of peptides in the same unit volume. Therefore, compared with the cell culture auxiliary agent using 4-methacryloxyethyl trimellitic anhydride as the linker, the cell culture auxiliary agent using maleic anhydride as the linker is more capable of allowing cells to aggregate and suspend.

Further, each peptide in the cell culture auxiliary agent of the present disclosure has cell affinity, and it can be selected from an oligopeptide composed of three to ten amino acids. In this embodiment, the amino acid sequences of the peptides are each independently selected from a group consisting of glycine-arginine-glycine-aspartate (Gly-Arg-Gly-Asp, GRGD), arginine-glycine-aspartate (Arg-Gly-Asp, RGD), arginine-glutamate-aspartate-valine (Arg-Glu-Asp-Val, REDV), leucine-aspartate-valine (Leu-Asp-Val, LDV), tyrosine-isoleucine-glycine-serine-arginine (Tyr-Ile-Gly-Ser-Arg, YIGSR), proline-aspartate-serine-glycine-arginine (Pro-Asp-Ser-Gly-Arg, PDSGR), isoleucine-lysine-valine-alanine-valine (Ile-Lys-Val-Ala-Val, IKVAV), and arginine-asparagine-isoleucine-alanine-glutamate-isoleucine-isoleucine-lysine-aspartate-alanine (Arg-Asn-Ile-Ala-Glu-Ile-Ile-Lys-Asp-Ala, RNIAEIIKDA), but the present disclosure is not limited thereto. In operation, an appropriate peptide can be selected according to the culture requirements of the desired cells. For instance, in order to form spherical aggregates after cell division, the cell culture auxiliary agent of the present disclosure can include GRGD peptides.

In certain embodiments, the implementable structural formula (P-L-S-L-P) of the cell culture auxiliary agent includes GRGD-MA-F127-MA-GRGD, GRGD-succinic anhydride-F127-succinic anhydride-GRGD, GRGD-4META-F127-4META-GRGD, RGD-MA-F127-MA-RGD, RGD-4META-F127-4META-RGD, and RGD-succinic anhydride-F127-succinic anhydride-RGD.

### Preparation Example

Take GRGD-MA-F127-MA-GRGD (hereinafter referred to as F127-Maleic-GRGD) as an example for preparing a cell culture auxiliary agent.

Firstly, referring to Formula (1), F127 (10 g, 0.787 mmol) is dissolved in 200 mL of anhydrous dichloromethane (DCM) to obtain a solution, and triethanolamine (TEA) (956 mg, 9.444 mmol) and maleic anhydride (MA) (617 mg, 6.296 mmol) are dropwise added to the solution under nitrogen at room temperature so as to obtain a mixture. After stirring the mixture at room temperature for 16 hours, the mixture is washed twice with water, and an organic layer is collected and dried by using magnesium sulfate (MgSO₄). The organic layer is filtered, and most of the solvent in the organic layer is removed by a rotary evaporator. Finally, excess of ether is used to precipitate the organic layer, and after collecting and filtering the precipitate, F127-Maleic anhydride (F127-Maleic) (67%) is obtained.

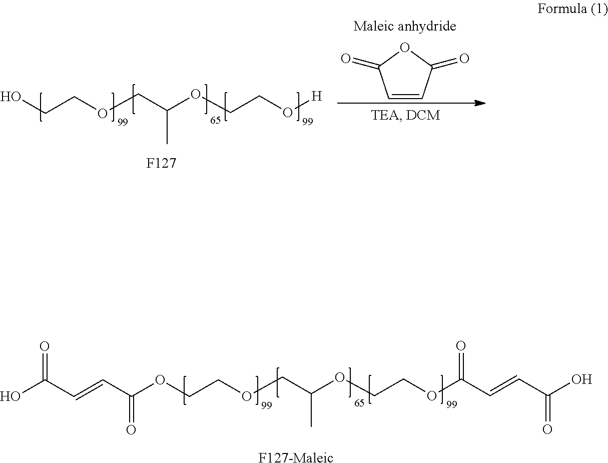

Next, referring to Formula (2), the obtained F127-Maleic (1.0 g, 0.078 mmol) is dissolved in 9 mL of tetrahydrofuran (THF) to obtain a solution, and N-hydroxysuccinimide (NHS) (72 mg, 0.624 mmol), N,N′-dicyclohexylcarbodiimide, (DCC) (129 mg, 0.624 mmol), and 4-dimethylaminopyridine (DMAP) are added to the solution under nitrogen at room temperature so as to obtain a mixture. After stirring the mixture at room temperature for 16 hours, the mixture is washed twice with water, and an organic layer is collected and dried by using magnesium sulfate (MgSO₄). The organic layer is filtered, and most of the solvent in the organic layer is removed by a rotary evaporator. Finally, excess of ether is used to precipitate the organic layer, and after collecting and filtering the precipitate, 640 mg of F127-Maleic anhydride-NHS (F127-Maleic-NHS) (64%) is obtained.

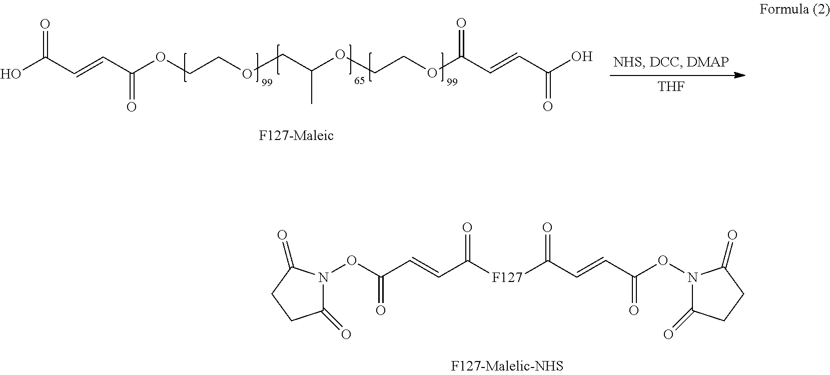

Finally, referring to Formula (3), the obtained F127-Maleic-NHS (500 mg, 0.039 mmol) and Gly-Arg-Gly-Asp (GRGD) peptides are dissolved in 3 mL of N,N-Dimethylformamide (DMF) to obtain a solution, and N,N-diisopropylethylamine (DIPEA) (25 mg, 0.195 mmol) is added to the solution under nitrogen at room temperature so as to obtain a mixture. After stirring the mixture at room temperature for 16 hours, the mixture is freeze-dried to remove DMF. Finally, cooled methanol is used to precipitate the mixture, and after collecting and filtering the precipitate, 295 mg of F127-Maleic-GRGD (59%) is obtained by high vacuum drying.

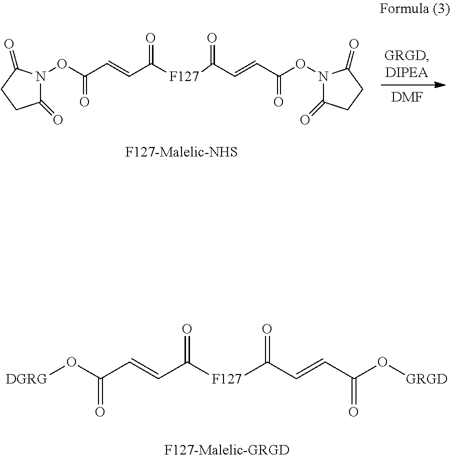

After stirring the mixture at room temperature for 16 hours, the mixture is washed twice with water, and an organic layer is collected and dried by using magnesium sulfate (MgSO₄). The organic layer is filtered, and most of the solvent in the organic layer is removed by a rotary evaporator. Finally, cooled methanol is used to precipitate the organic layer, and after collecting and filtering the precipitate, F127-Maleic anhydride (F127-Maleic) (67%) is obtained.

However, the aforementioned description for the cell culture auxiliary agent of the preparation example is merely an example and is not meant to limit the scope of the present disclosure.

### Second Embodiment

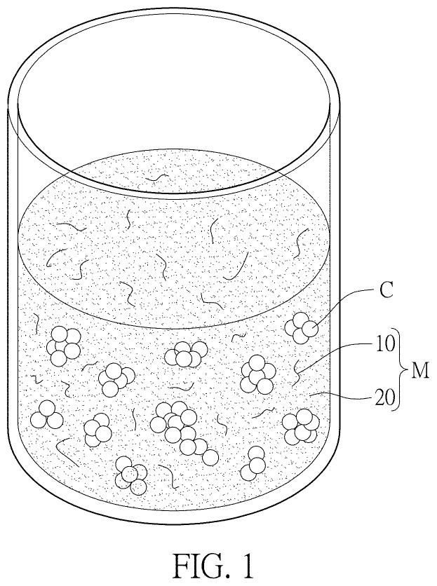

Referring to FIG. 1, a second embodiment of the present disclosure provides a cell culture medium M including 0.5-5 wt % of the cell culture auxiliary 10 as described in the first embodiment and 10-99.5 wt % of a cell culture solution 20. The cell culture medium M of the present disclosure can provide cells C a 3D environment culture environment that allows the cells C to grow in a dispersed and suspended state, and aggregate in a spherical shape after division.

Further, the cell culture solution can be based on, but not limited to, isotonic solutions such as balanced salt solution (BSS), phosphate buffered saline (PBS), and in which further nutrients are added (such as amino acids, vitamins, serum, and so on) and/or antibiotics required by the cells to be cultured.

In certain embodiments, the cell culture solution can be minimal essential medium (MEM), Dulbecco's modified minimal essential medium (DMEM), Roswell Park Memorial Institute-1640 medium (RPMI-1640), Iscove's modified DMEM (IMDM), or serum free medium (SFM), but the present disclosure is not limited thereto.

In certain embodiments, the cell culture medium can be prepared by mixing the cell culture auxiliary into a commercially available basal minimal essential medium (MEM), for instance, 1 ml of cell culture auxiliary dispersion can be mixed with 99 ml of basal medium to prepare cell culture medium, but the present disclosure is not limited thereto.

It should be noted that, in operation, since Pluronic® F-127 is a temperature-sensitive material, it can form a hydrogel-like microstructure carrier in a cell culture medium at a temperature close to body temperature (about 37° C.) so as to carry cells and keep the cells in a dispersed and suspended state in the cell culture solution. After the cell culture is completed, the cell culture medium can be placed in a low temperature environment to make the microstructure carrier return to a liquid state to facilitate cell collection or replacement of the cell culture substrate.

### Third Embodiment

Human hair follicle mesenchymal stem cells (hfMSCs) were cultured for 7 days using the cell culture medium containing different concentrations of the cell culture auxiliary of the present disclosure, and their aggregation patterns and cell viability were observed.

The cell culture medium was divided into five groups, namely control group (C), experimental group (1), experimental group (2), experimental group (3) and experimental group (4). Among them, the cell culture solutions of all groups are all commercially available basal media. The difference between the groups is that the concentration of the cell culture auxiliary in the cell culture medium is different. Specifically speaking, the concentration of the cell culture auxiliary in the cell culture medium of the control group (C) was 0%; the concentration of the cell culture auxiliary in the cell culture medium of the experimental group (1) was 0.05%; the concentration of the cell culture auxiliary in the cell culture medium of the experimental group (2) was 0.1%; the concentration of the cell culture auxiliary in the cell culture medium of the experimental group (3) was 0.25%; and the concentration of the cell culture auxiliary in the cell culture medium of the experimental group (4) was 0.5%.

1×10⁶ cells of hfMSCs were seeded into 96-well cell dishes, which contained cell culture medium with different cell growth aid concentrations, and the 96-well cell dishes were placed in an incubator at 37° C. and a carbon dioxide concentration of 5% for 7 days, and the cell culture media were changed every 2 days. Among them, the cell aggregation pattern, the cell viability and proliferation rate were observed on the first day, the fourth day, and the seventh day.

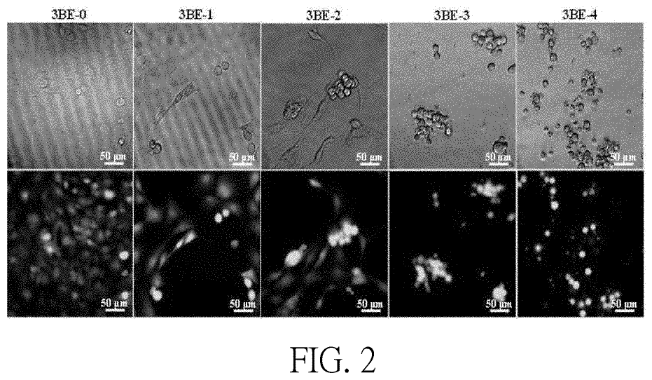

The cells were fluorescently stained, where green fluorescence was live cells and red fluorescence was dead cells. Referring to FIG. 2, as the concentration of the cell culture auxiliary increases, the cells exhibit a better effect of spherical aggregation.

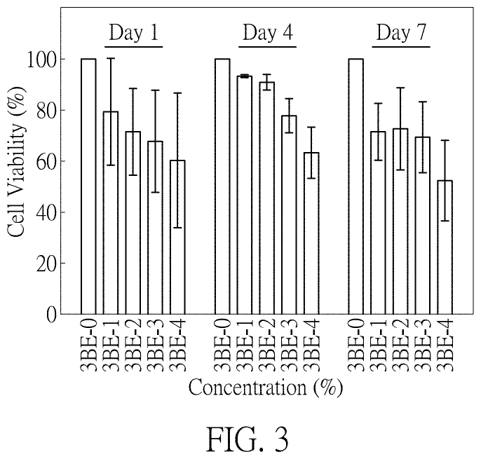

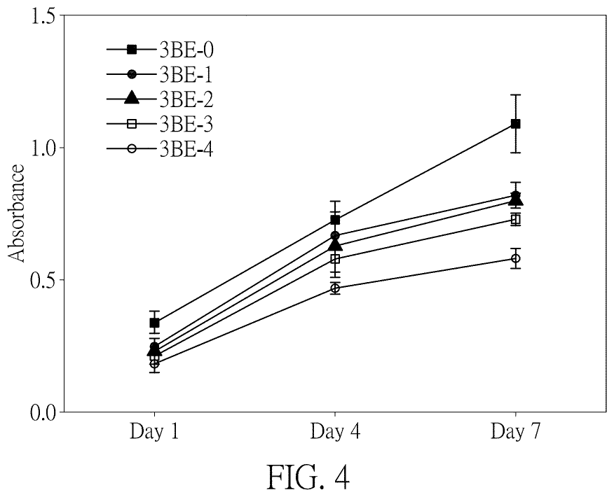

Referring to FIG. 3 and FIG. 4, each experimental group containing the cell culture auxiliary had an average cell viability of 70%.

The results of this embodiment show that because the cell culture medium of the present disclosure contains the cell culture auxiliary, it can create a three-dimensional culture environment in vitro, help cells grow in a three-dimensional aggregation manner, and achieve the effect of improving cell proliferation and survival.

### Fourth Embodiment

Michigan cancer foundation-7 cells (MCF-7) were cultured for 7 days using the cell culture medium containing different concentrations of the cell culture auxiliary of the present disclosure, and their aggregation patterns and cell viability were observed.

The cell culture medium was divided into five groups, namely control group (C), experimental group (1), experimental group (2), experimental group (3) and experimental group (4). Among them, the cell culture solutions of all groups are all commercially available basal media. The difference between the groups is that the concentration of the cell culture auxiliary in the cell culture medium is different. Specifically speaking, the concentration of the cell culture auxiliary in the cell culture medium of the control group (C) was 0%; the concentration of the cell culture auxiliary in the cell culture medium of the experimental group (1) was 0.05%; the concentration of the cell culture auxiliary in the cell culture medium of the experimental group (2) was 0.1%; the concentration of the cell culture auxiliary in the cell culture medium of the experimental group (3) was 0.25%; and the concentration of the cell culture auxiliary in the cell culture medium of the experimental group (4) was 0.5%.

5×10³ cells of MCF-7 were seeded into 96-well cell dishes, which contained cell culture medium with different cell growth aid concentrations, and placed the 96-well cell dishes in an incubator at 37° C. and a carbon dioxide concentration of 5% for 7 days, and the cell culture media were changed every 2 days. Among them, the cell aggregation pattern, the cell viability and proliferation rate were observed on the first day, the fourth day, and the seventh day.

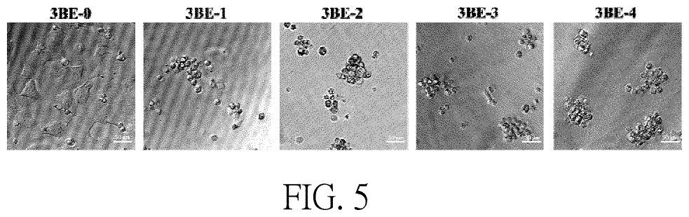

Referring to FIG. 5, as the concentration of the cell culture auxiliary increases, the cells exhibit a better effect of spherical aggregation.

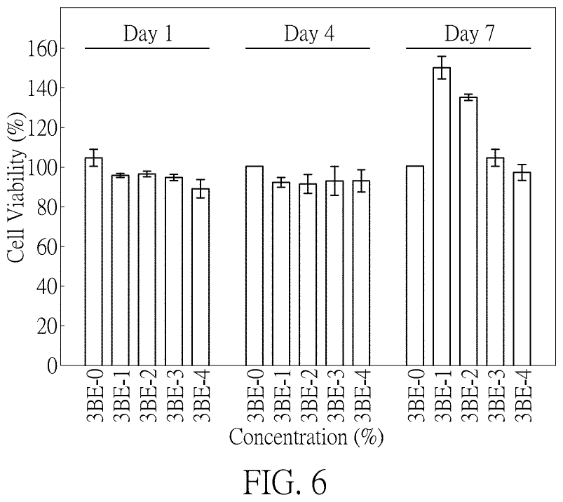

Referring to FIG. 6, each experimental group containing the cell culture auxiliary had an average cell viability of 70%.

The results of this embodiment show that because the cell culture medium of the present disclosure contains the cell culture auxiliary, it can create a three-dimensional culture environment in vitro, help cells grow in a three-dimensional aggregation manner, and achieve the effect of improving cell proliferation and survival.

### Beneficial Effects of the Embodiments

In conclusion, by virtue of “the cell culture auxiliary agent is formed by attaching each coordination peptide having cell affinity to two side ends of a polyoxyethylene polyoxypropylene ether block copolymer through anhydride monomers”, the cell culture auxiliary agent of the present disclosure can form a microstructure carrier that allows cells to attach in the cell culture medium, and allows the cells to grow in a suspended state in the cell culture medium, thereby improving cell proliferation and survival rate.

Furthermore, because the polyoxyethylene polyoxypropylene ether block copolymer is temperature-sensitive, the cell culture substrate of the present disclosure can form a hydrogel-like microstructure carrier at room temperature (about 25° C.) or culture temperature (about 37° C.), which can carry cells and keep the cells in a dispersed and suspended state, and then aggregate in a spherical shape after division. It should be noted that the microstructure carriers can be turned back into liquid at low temperature (below 10° C.), which is convenient for cell collection or replacement of cell culture medium. Therefore, the cell culture medium of the present disclosure can provide cells cultured in vitro having basically the same growth conditions as cells cultured in vivo, and make the operation more convenient and flexible for experimenters; and can reduce the experiment cost because no additional 3D colloid is required.

Moreover, the cell culture auxiliary agent of the present disclosure uses anhydride monomers to link polyoxyethylene polyoxypropylene ether block copolymers with cell-affinity peptides, which can improve structural stability, and the peptides can more easily help cells aggregate and suspend.

The foregoing description of the exemplary embodiments of the disclosure has been presented only for the purposes of illustration and description and is not intended to be exhaustive or to limit the disclosure to the precise forms disclosed. Many modifications and variations are possible in light of the above teaching.

The embodiments were chosen and described in order to explain the principles of the disclosure and their practical application so as to enable others skilled in the art to utilize the disclosure and various embodiments and with various modifications as are suited to the particular use contemplated. Alternative embodiments will become apparent to those skilled in the art to which the present disclosure pertains without departing from its spirit and scope.

What is claimed is:

1. A cell culture auxiliary agent comprising a structural formula represented as follow: P-L-S-L-P; wherein in the structural formula, S is a substrate, each L is a linker, and each P is a peptide; and wherein the substrate is a polyoxyethylene polyoxypropylene ether block copolymer, the linkers are each independently an anhydride monomer, and an amino acid sequence of the peptides are each independently selected from a group consisting of glycine-arginine-glycine-aspartate, arginine-glycine-aspartate, arginine-glutamate-aspartate-valine, leucine-aspartate-valine, tyrosine-isoleucine-glycine-serine-arginine, proline-aspartate-serine-glycine-arginine, isoleucine-lysine-valine-alanine-valine, and arginine-asparagine-isoleucine-alanine-glutamate-isoleucine-isoleucine-lysine-aspartate-alanine.

2. The cell culture auxiliary agent according to claim 1, wherein the substrate is Pluronic® F-127, and the anhydride monomer is maleic anhydride, succinic anhydride, or 4-methacryloxyethyl trimellitic anhydride.

3. The cell culture auxiliary agent according to claim 2, wherein the anhydride monomer is maleic anhydride, and the amino acid sequence of the peptide is glycine-arginine-glycine-aspartate.

4. The cell culture auxiliary agent according to claim 2, wherein the anhydride monomer is 4-methacryloxyethyl trimellitic anhydride, and the amino acid sequence of the peptide is glycine-arginine-glycine-aspartate.

5. A cell culture medium, which comprises 0.5-5 wt % of a cell culture auxiliary agent including a structural formula represented as follow: P-L-S-L-P; wherein in the structural formula, S is a substrate, each L is a linker, and each P is a peptide; and wherein the substrate is a polyoxyethylene polyoxypropylene ether block copolymer, the linkers are each independently an anhydride monomer, and an amino acid sequence of the peptides are each independently selected from a group consisting of glycine-arginine-glycine-aspartate, arginine-glycine-aspartate, arginine-glutamate-aspartate-valine, leucine-aspartate-valine, tyrosine-isoleucine-glycine-serine-arginine, proline-aspartate-serine-glycine-arginine, isoleucine-lysine-valine-alanine-valine, and arginine-asparagine-isoleucine-alanine-glutamate-isoleucine-isoleucine-lysine-aspartate-alanine.

6. The cell culture medium according to claim 5, further comprising 95-99.5 wt % of a cell culture solution.

7. The cell culture medium according to claim 6, wherein the cell culture auxiliary agent is applied to make cells being suspended in the cell culture medium.

8. The cell culture medium according to claim 5, wherein the substrate is Pluronic® F-127, and the anhydride monomer is maleic anhydride, succinic anhydride, or 4-methacryloxyethyl trimellitic anhydride.

9. The cell culture medium according to claim 8, wherein the anhydride monomer is maleic anhydride, and the amino acid sequence of the peptide is glycine-arginine-glycine-aspartate.

10. The cell culture medium according to claim 8, wherein the anhydride monomer is 4-methacryloxyethyl trimellitic anhydride, and the amino acid sequence of the peptide is glycine-arginine-glycine-aspartate.
